(19) United States
(12) Patent Application Publication
(10) Pub. No.: US 2026/0053584 A1
(43) Pub. Date: Feb. 26, 2026

(54) METHOD FOR CONTROLLING A SLAVE DEVICE, CONTROLLED BY A MASTER DEVICE IN A ROBOTIC SYSTEM FOR MEDICAL OR SURGICAL TELEOPERATION, TAKING INTO ACCOUNT LIMITS OF A FIELD OF VIEW, AND RELATED ROBOTIC SYSTEM

(71) Applicant: MEDICAL MICROINSTRUMENTS, INC., Wilmington, DE (US)

(72) Inventors: Massimiliano SIMI, Pisa (IT); Emanuele RUFFALDI, Pisa (IT); Antonio DI GUARDO, Pisa (IT)

(21) Appl. No.: 19/102,822

(22) PCT Filed: Aug. 9, 2023
(86) PCT No.: PCT/IB2023/058043

§ 371 Date: Feb. 10, 2025

(30) Foreign Application Priority Data
Aug. 10, 2022 (IT) 102022000017103

(51) Int. Cl. A61B 34/35; A61B 90/00

(52) U.S. Cl. CPC A61B 34/35; A61B 90/361; A61B 2090/373

(57) ABSTRACT

A method controls a slave device of a robotic system for medical or surgical teleoperation. The robotic system includes a master device movable by an operator, a slave device having a surgical instrument controllable by the master device, and a viewer configured to display to the operator images and/or videos of a viewing space associated with a teleoperation area in which the surgical instrument operates. Position of the surgical instrument is determined with respect to the viewing space, to determine whether the surgical instrument is located inside or outside an allowed space correlated to the viewing space. Movement of the slave device is dependent on the determined position of the surgical instrument with respect to the allowed space correlated to the viewing space, and so that movement of the surgical instrument is allowed only if located inside the viewing space. A robotic system is controllable by the control method.

### CROSS-REFERENCE TO RELATED APPLICATIONS

This application is a National Stage Application of PCT/IB2023/058043, filed Aug. 9, 2023, which claims benefit of priority to Italian Patent Application No. 102022000017103 filed on Aug. 10, 2022, and which applications are incorporated herein by reference. To the extent appropriate, a claim of priority is made to each of the above disclosed applications.

### FIELD OF APPLICATION

The present invention relates to a method and system for controlling a robotic system for medical or surgical teleoperation.

In particular, the invention relates to a method for controlling a slave device, controlled by a master device in a robotic system for medical or surgical teleoperation, taking into account limits of a field of view, and the related robotic system.

### PRIOR ART

In a system for robotic surgery, the field of view (FOV) provided by any associated viewing system associated therewith (endoscope, laparoscope, microscope or exoscope) is typically included in the workspace of the slave device (also defined a “slave workspace”).

In other words, often, due to a high use of magnification, or a position of the camera which is very close to the work area, or a small workspace, or simply due to a large workspace of the slave device, the field of view FOV is a subspace, i.e., it represents a subset of the workspace of the joints of the slave device.

Therefore, a movement of an instrument controlled by the master device can be mapped inside the slave workspace (i.e., inside the space of the slave joints) but outside the effective field of view FOV and therefore is not carried out under the complete control of the operator who, in a robotic teleoperation system, closes the control loop of each movement through his own view mediated by the viewing system.

In particular, carrying an instrument out of the field of view, or moving an instrument when already out of the field of view, can be dangerous and potentially cause damage to the patient, such as perforations and/or lacerations of tissues. This is because the robotic instruments are usually much stiffer, stronger, or sharper than the tissue can withstand.

A further risk can arise from such a situation, deriving from the operator's attempt to re-enter teleoperation with the instruments outside the FOV or once they have exited, aligning or moving the instrument “blindly” when this is no longer in the field of view and thus greatly increasing the risk of damage to the patient.

A method is known from US 2022 000579 which provides, in a robotic system, for the autonomous backwards movement of the endoscope when the instrument exits the field of view, so as to bring the instrument back into the FOV, widening the field. In particular, the instrument is maintained in the field of view by autonomously rotating (roll) the laparoscopic camera which has an FOV inclined by an angle (e.g., 30° or 45°) from the top of the endoscope, so as to achieve, rotating for example by a full turn, a panoramic view of the surgical site.

This solution is prone to some drawbacks such as, for example, discomfort due to the frequent movements of the viewing system (position and/or orientation) to follow the surgical instrument, as well as the delay in updating the panoramic image, and/or the corresponding frequent changes visible on the screen as a result of the repositioning of the camera which could disorient an operator during surgery.

It is known from US 2018 0025666 to control the camera, to move it, using for example the master controller during a suspended teleoperation state.

The known solutions, in the technical field considered, do not allow satisfactorily solving the aforesaid problems and drawbacks.

In particular, the need to avoid the risk of causing harm to the patient when the surgical instrument is not visible remains felt. In addition, the risk of causing damage to the patient also manifests if a certain portion of the patient's anatomy is not visible because it is outside the field of view and the surgical instrument is moved towards it.

These disadvantageous effects are particularly felt in those robotic platforms without force feedback on the master device, as well as those which avoid including mechanical constraints to the movements of the master device, as well as those intended for mono-lateral teleoperation.

Therefore, in the technical field considered, there is a strong need to control the enslaved movement of the slave device, depending on the master device, with expedients and based on control algorithms such as to solve or at least mitigate the aforesaid problems and drawbacks.

### SUMMARY OF THE INVENTION

It is an object of the present invention to provide a method for controlling a slave device, controlled by a master device in a robotic system for medical or surgical teleoperation, taking into account limits of a field of view, which allows at least partially obviating the drawbacks complained above with reference to the prior art, and responding to the aforementioned needs particularly felt in the technical field considered.

It is also an object of the present invention to provide a robotic system for medical or surgical teleoperation, configured to be controlled by the aforesaid method.

### BRIEF DESCRIPTION OF THE DRAWINGS

Further features and advantages of the method according to the invention will become apparent from the following description of preferred embodiments, given by way of non-limiting indication, with reference to the accompanying drawings, in which:

FIGS. 1 and 2 show two respective embodiments of a robotic system for medical or surgical teleoperation according to the invention;

FIG. 3 shows two surgical instruments included in the aforesaid robotic system in relation to a space in which they must operate, according to an embodiment of the method according to the invention;

FIG. 4 shows a surgical instrument included in the aforesaid robotic system, and possible movements thereof in relation to a space in which it must operate, according to an embodiment of the method according to the invention;

FIGS. 5, 5bis and 5ter depict respective simplified block diagrams of a robotic system of the invention, according to respective embodiments;

FIGS. 6, 7, 7bis, 7ter, 7quater, 8, 9, 10, 11, 12, 13, 14, 16 and 16bis show respective surgical instruments included in the aforesaid robotic system in relation to different spaces in which they must operate, correlated to viewing spaces, according to respective embodiments of the method according to the invention;

FIG. 15 shows geometric aspects of a viewing space defined by an embodiment of the method according to the invention.

### DETAILED DESCRIPTION

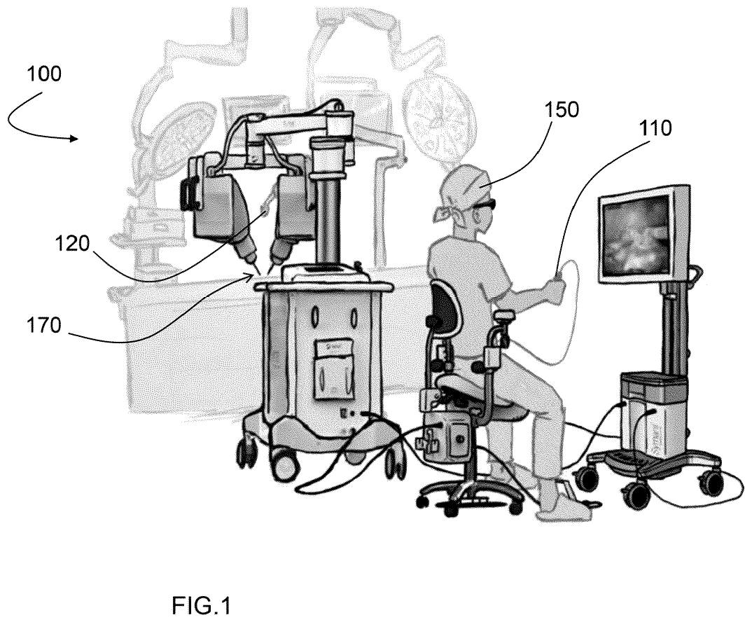

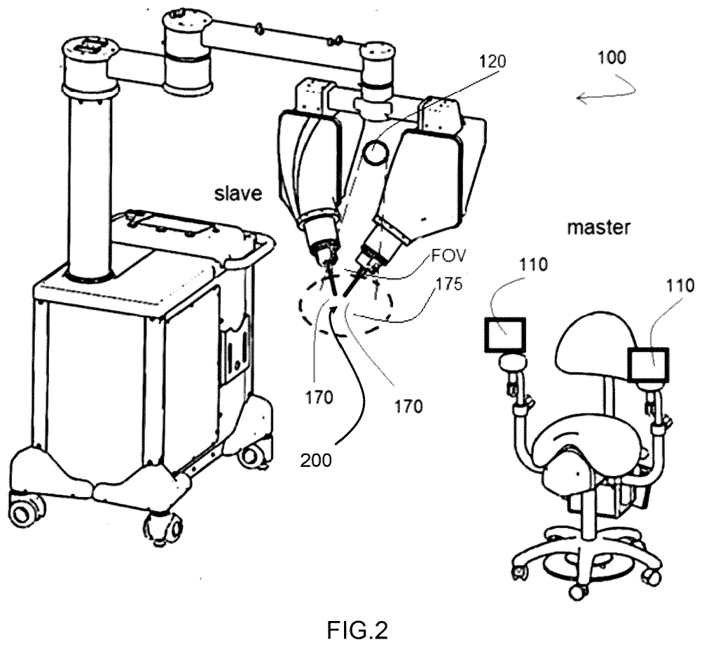

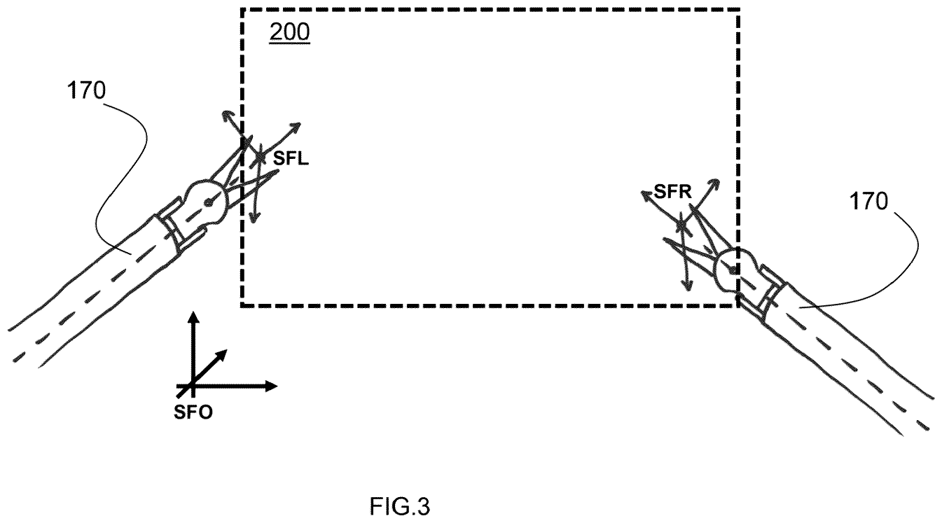

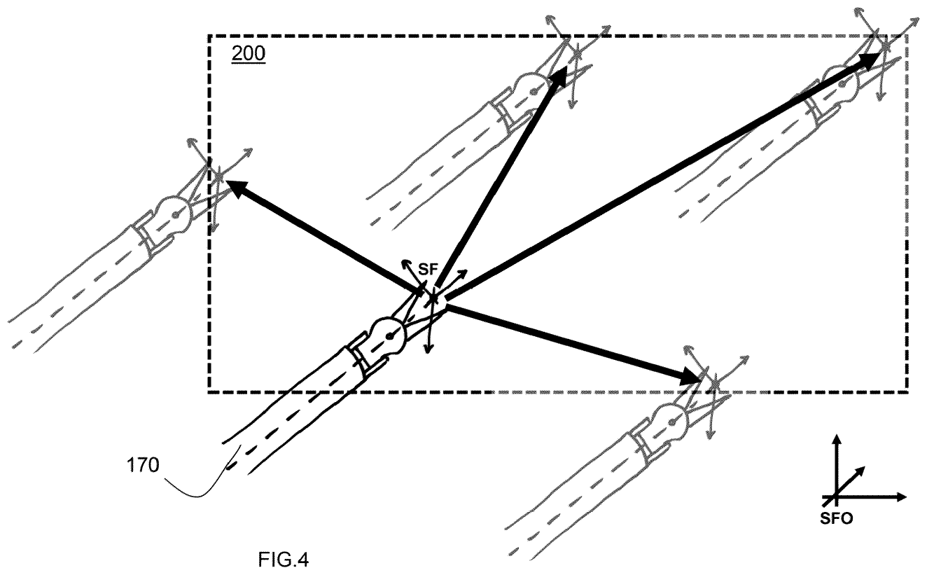

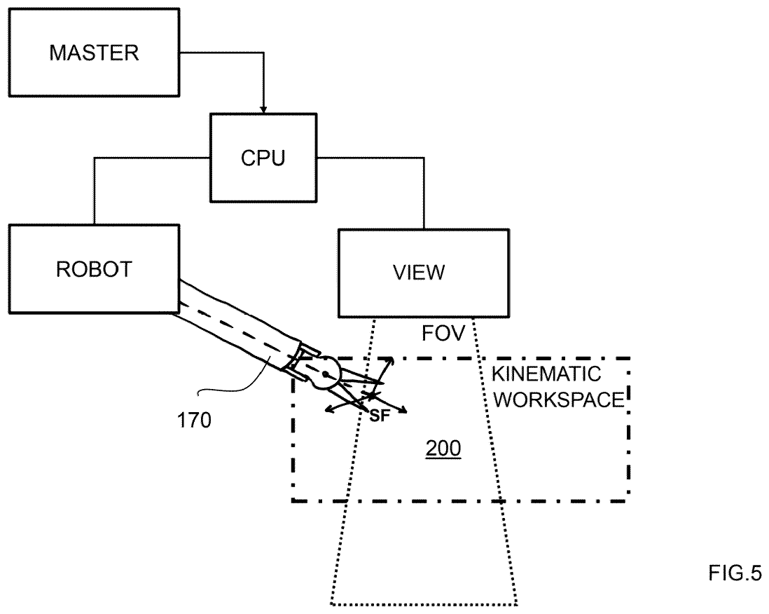

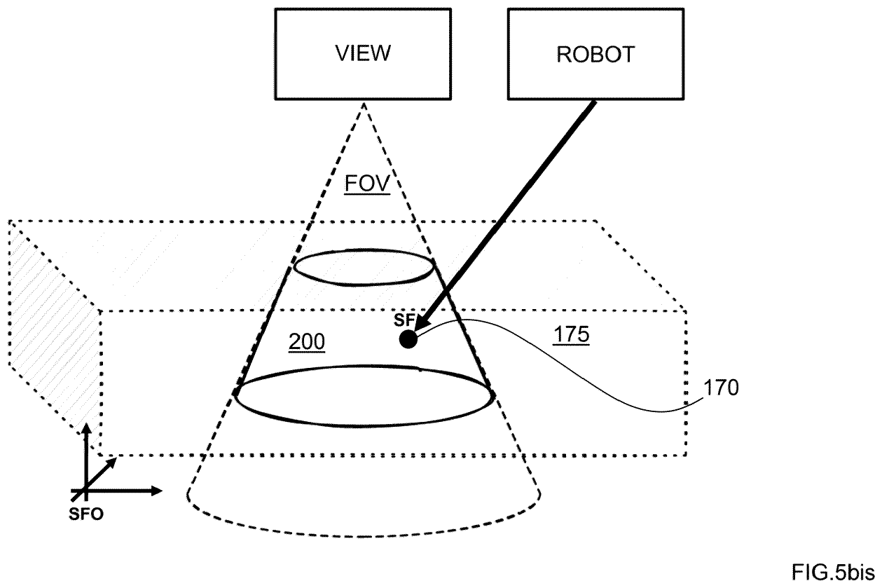

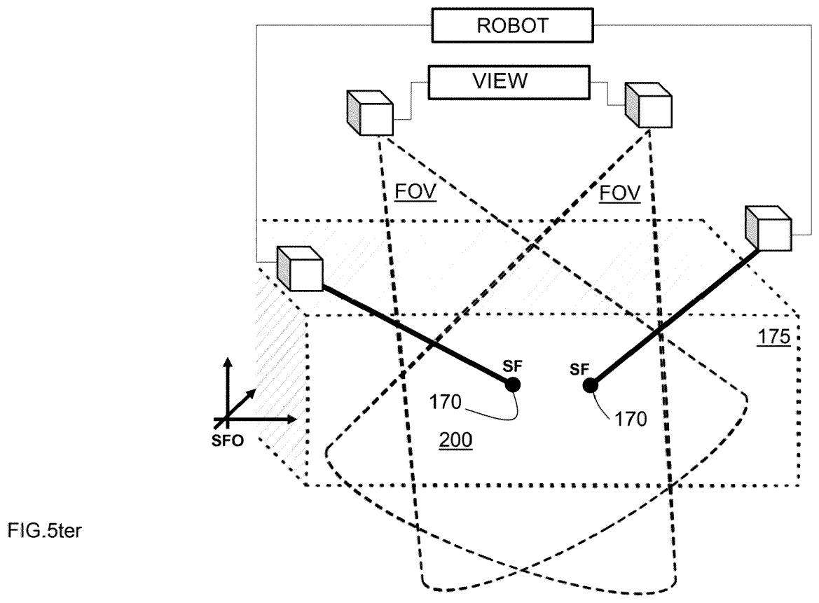

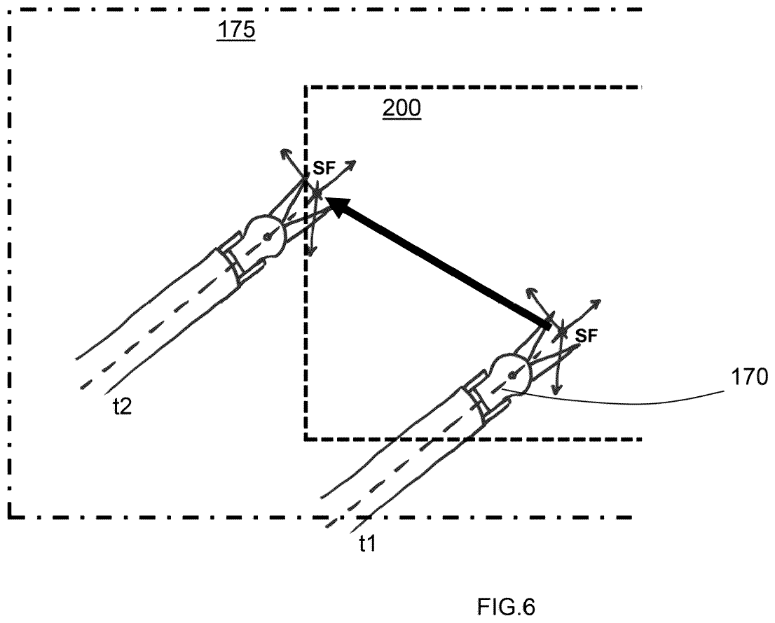

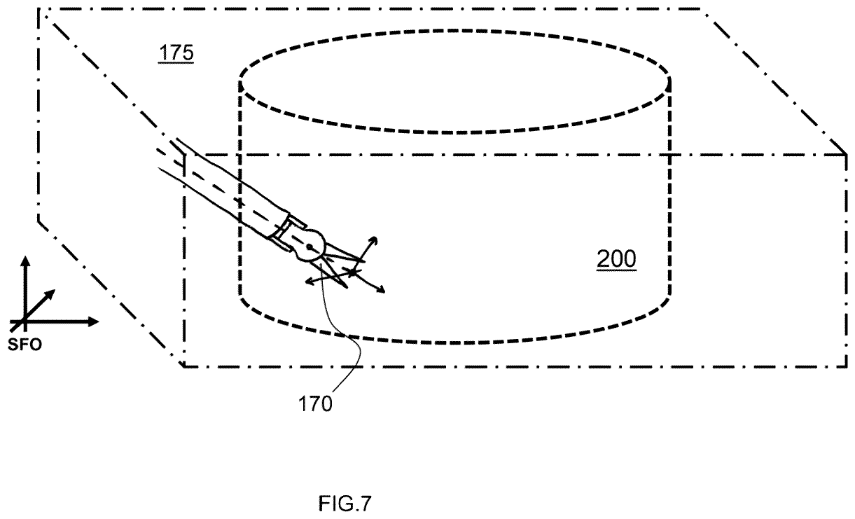

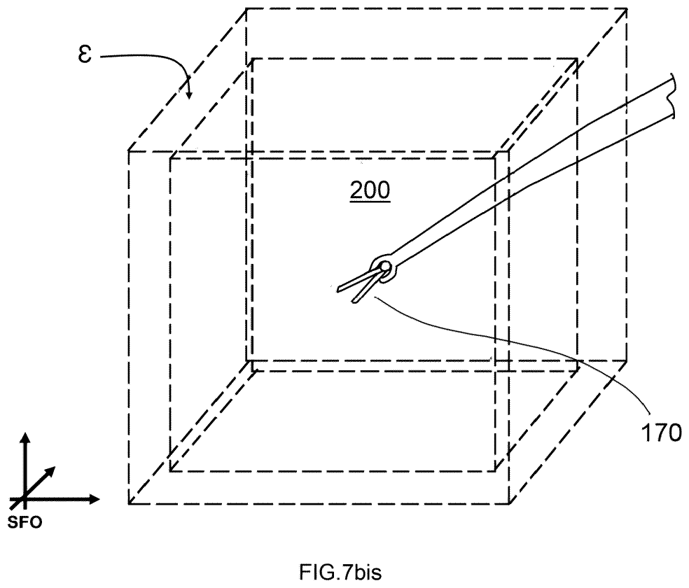

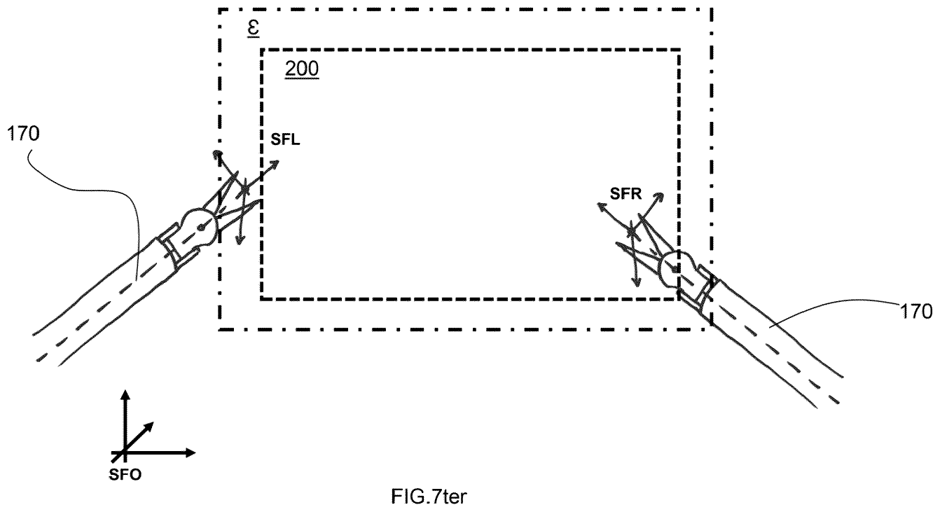

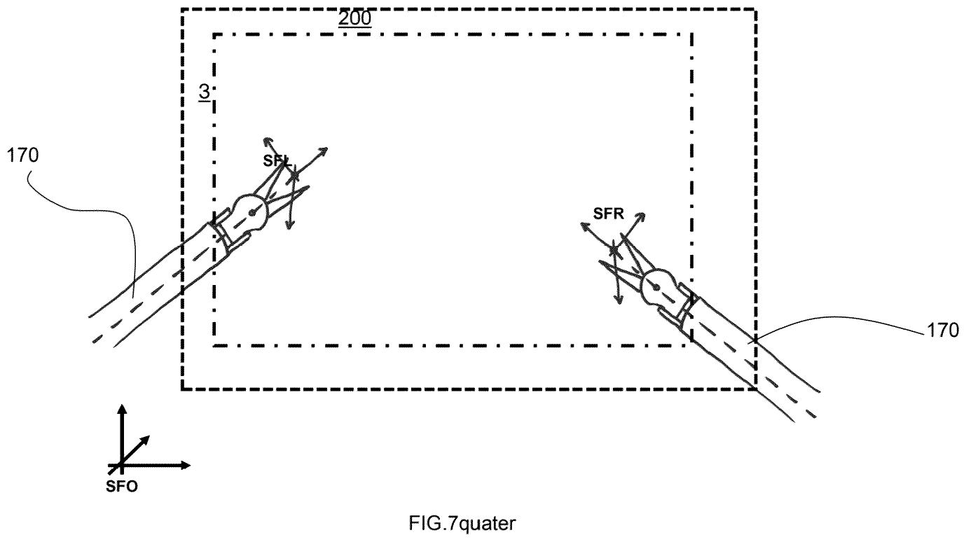

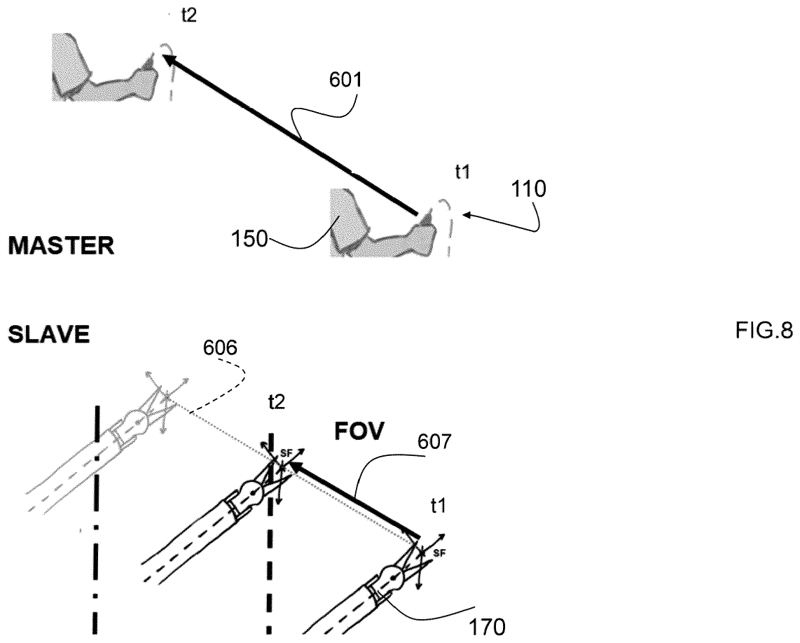

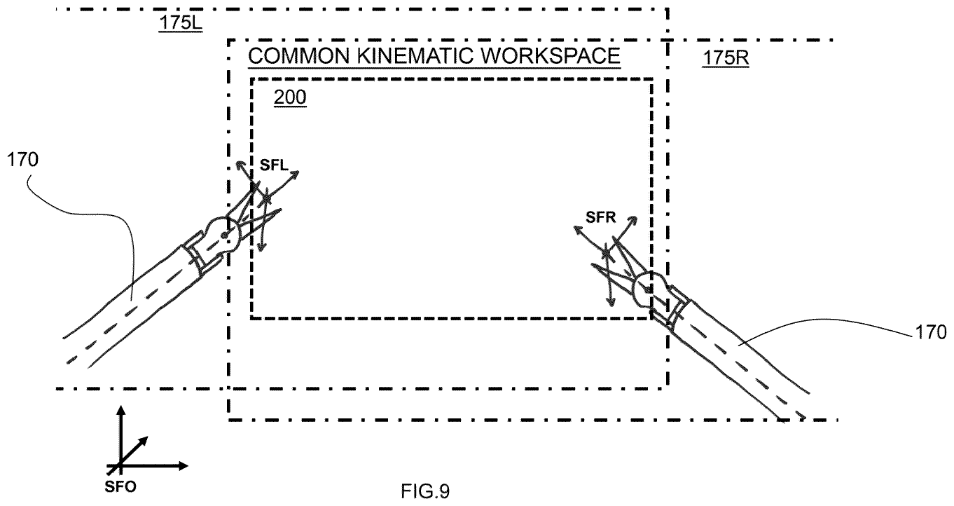

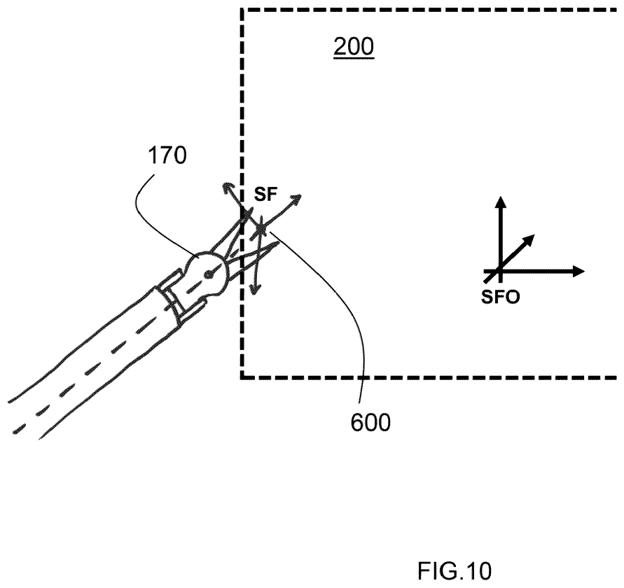

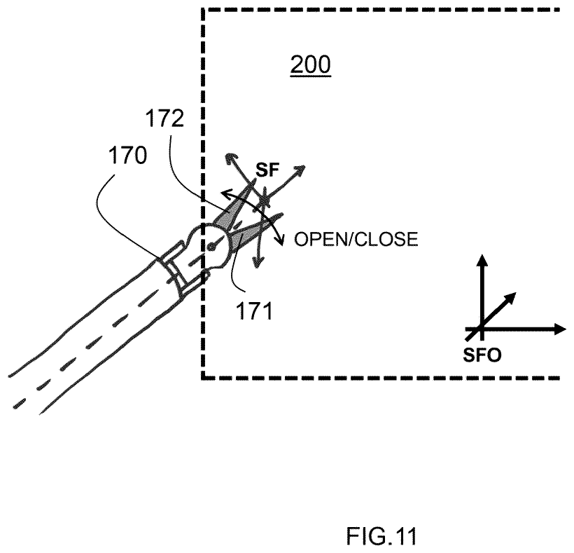

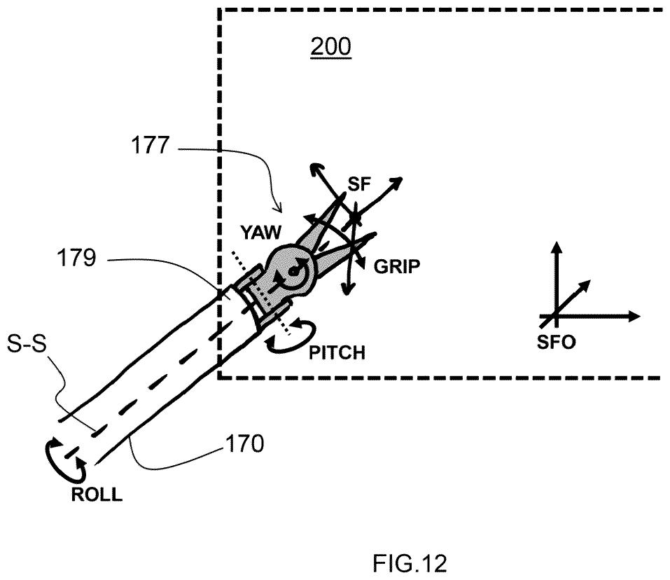

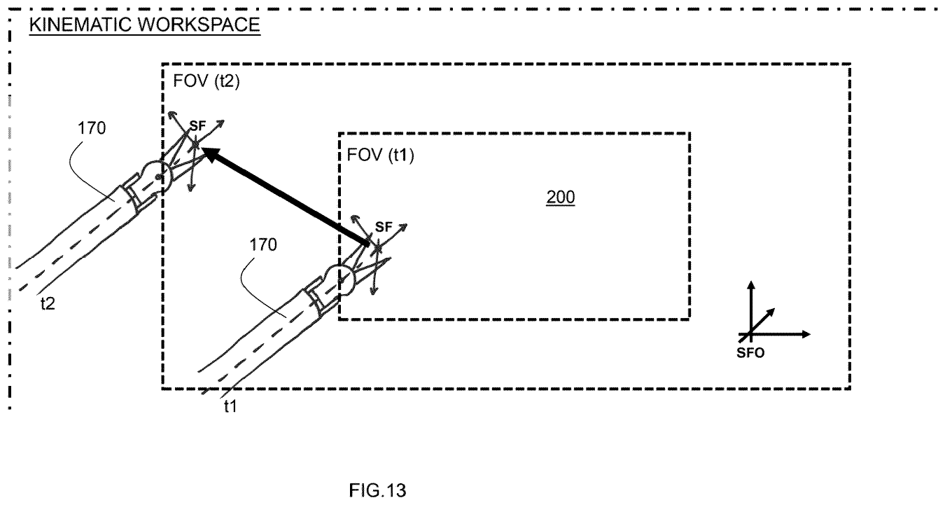

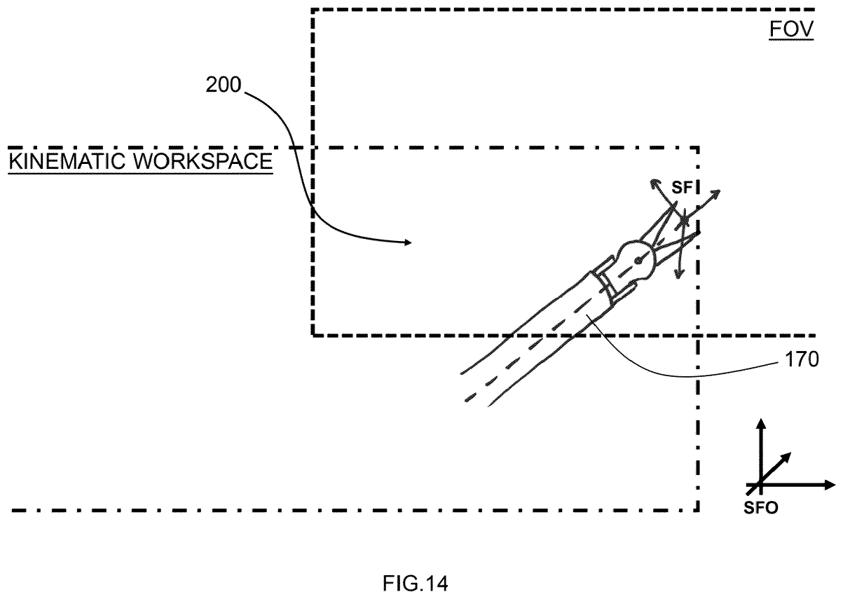

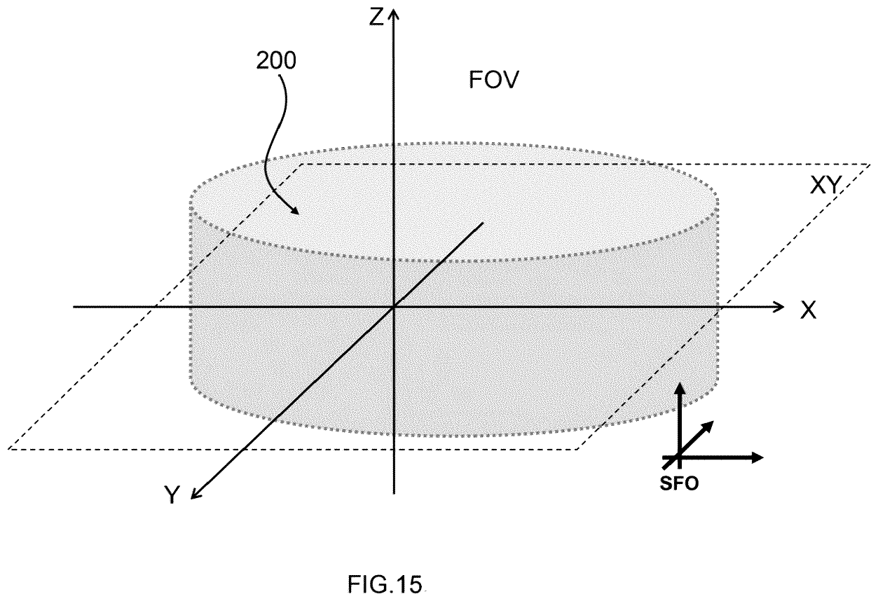

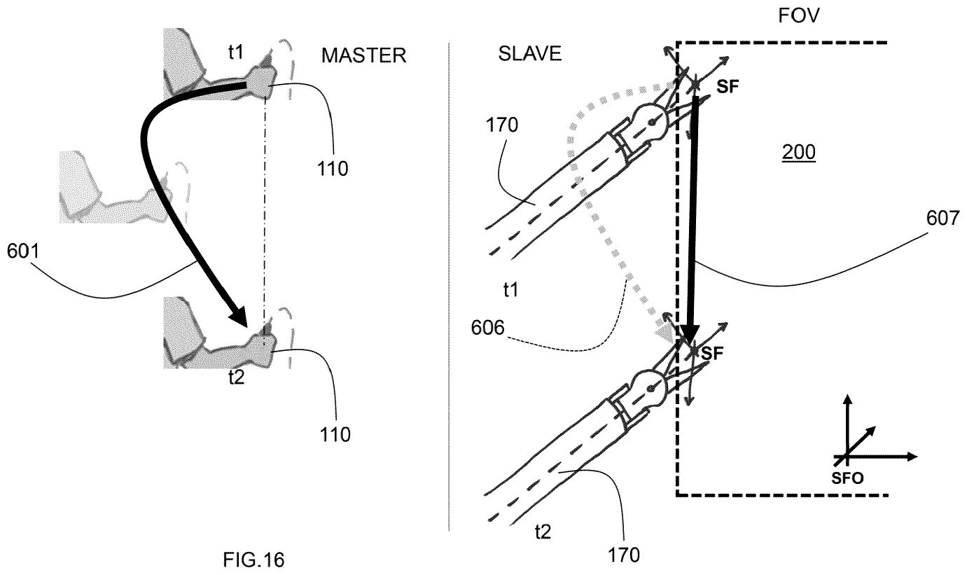

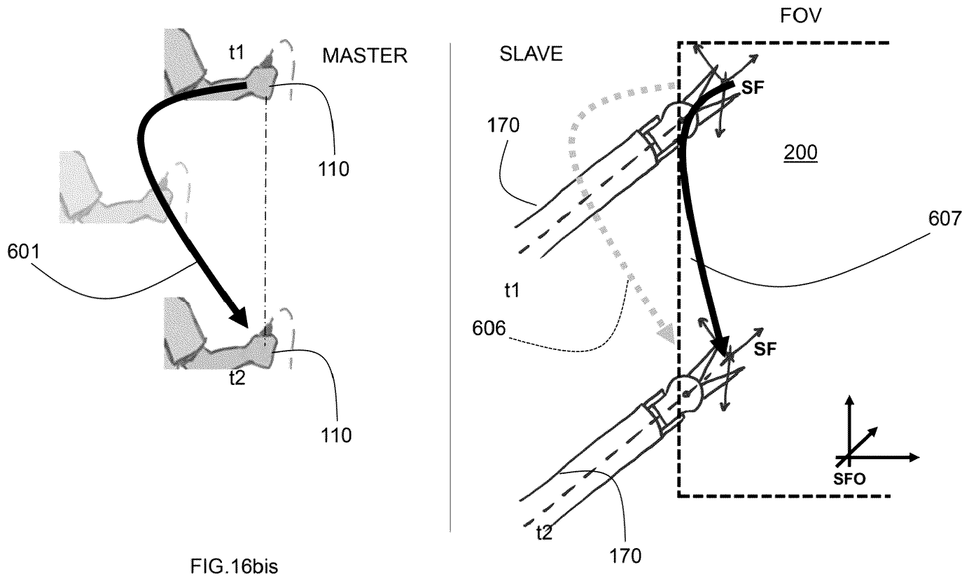

With reference to FIGS. 1-16, a method for controlling a slave device of a robotic system for medical or surgical teleoperation is described.

The robotic system, to which the method is applied, comprises at least one master device 110 adapted to be moved by an operator 150, at least one slave device comprising a surgical instrument 170 adapted to be controlled by the master device, and further comprises viewing means configured to display to the operator 150 images and/or videos of a viewing space associated with a teleoperation area in which the surgical instrument 170 operates.

The master device 110 is preferably an “ungrounded” type master device, without force feedback, for mono-lateral teleoperation. For example, therefore, the master device can be a master mechanically constrained to an operating console and at the same time be of the “ungrounded” type without force feedback, for mono-lateral teleoperation.

The master device 110 is preferably a master device of a type which is mechanically unconstrained to the operating console.

The method first comprises a step of determining a position of the surgical instrument 170 with respect to said viewing space, to determine whether the surgical instrument 170 is located inside or outside an allowed space correlated to the viewing space.

The method then provides controlling the movement of the slave device in a manner dependent on the determined position of the surgical instrument 170 with respect to the allowed space correlated to the viewing space, and so that the movement of the surgical instrument 170 is allowed only if the surgical instrument 170 is located inside the allowed space correlated to the viewing space, and that the movement of the surgical instrument 170, if allowed, is in any case confined within the aforesaid viewing space.

According to an implementation option, the method includes that the movement of the surgical instrument 170, if allowed, is in any case confined within the aforesaid allowed space correlated to the viewing space.

According to an embodiment of the method, the aforesaid allowed space correlated to the viewing space corresponds to the viewing space.

According to another embodiment of the method, the aforesaid allowed space correlated to the viewing space comprises the viewing space and further comprises an external surrounding extending with a spatial tolerance e beyond the boundaries of the viewing space.

According to another embodiment of the method, the aforesaid allowed space correlated to the viewing space comprises a subset of the viewing space, corresponding to the viewing space from which an internal surrounding extending with a spatial tolerance e inside the boundaries of the viewing space is removed.

According to an implementation option of the aforesaid embodiments which include a spatial tolerance, said step of determining whether the surgical instrument 170 is located inside or outside the viewing space is carried out within the tolerance of such a spatial tolerance e, near the boundaries of the viewing space.

According to an implementation option of the method, the aforesaid viewing space is defined by a field of view (FOV) of the viewing means.

Such an implementation refers to a robotic system having viewing means, or a generic viewing system (comprising digital image/video acquisition means), capable of capturing a portion of the world observed through appropriate lens or light guide systems.

With a terminology known in the technical field considered, such a portion of the world of which an image or video is acquired has an extension referred to as the “Field of View” (FOV) which is typically represented in angular units taken along the diagonal or one of the axes of the digital image/video acquisition system.

According to another implementation option of the method, said viewing space is defined by a predefined subset of the field of view (FOV) of the viewing means.

In fact, the boundaries of the area or volume defining the viewing space of interest do not necessarily coincide with the field of view; such boundaries can be constructed on a sub-volume of the field of view where the view is optimal and/or to have a particular geometric shape and/or specially chosen to promote the mobility of the slave device therein and/or for any other reason.

According to another implementation option of the method, the aforesaid viewing space is defined by a field-of-view workspace, consisting of a geometric volume, in a reference coordinate system of the robotic system, associated with the aforesaid field of view.

Such a field-of-view workspace (hereinafter also referred to as “FOV Workspace”) can for example correspond to a volume, for example a trapezoid which goes from the lens to infinity and centered in the main axis of the optical system, which is capable of representing the field of view of a digital viewing system, for example for lenses with “Fields of View” FOV less than 180 degrees. Once a plane is fixed with respect to the lens, it is possible to evaluate the extension of the field of view in metric terms by evaluating the portion of the plane which intersects the “FOV Workspace”, and generally such a plane is orthogonal to the main axis. The diagonal of the rectangle of said plane at a certain distance can be defined as “FOV Diagonal”.

According to another implementation option of the method, the aforesaid viewing space is defined by geometric limits of the field of view, consisting of a boundary surface of the aforesaid viewing workspace, in the reference coordinate system of the robotic system.

For example, the field-of-view workspace is constructed with respect to the trapezoid originating in the camera image plane of the viewing system. It is possible to construct simplified geometries therefrom, referred to as “field-of-view workspace limits” (FOV Workspace Limits) which are imposed to limit the movement of the slave device. Such geometries can be defined as planes orthogonal to the viewing system or as curved surfaces which in any case are defined within the field-of-view workspace.

According to several possible embodiments, the aforesaid viewing means comprise at least one camera 120 or comprise an endoscope and/or a laparoscope and/or a microscope and/or an exoscope.

According to an implementation, the viewing means comprise a stereoscopic viewing system comprising two cameras, each of which defines a respective “FOV Workspace” (175L, 175R), referred to as the “field-of-view workspace of camera L” (FOV Workspace L) and “field-of-view workspace of camera R” (FOV Workspace R). The intersection of the aforesaid two field-of-view workspaces of camera L and R produces a “common field-of-view workspace” which ensures the maximum visibility of the objects in the scene.

For a given point in such a “common field-of-view workspace”, the disparity or difference (in a given unit) of the lateral position of the same element can be calculated. Excessive disparity can lead to lack of depth perception and thus a blurring effect.

According to different possible embodiments, the aforesaid viewing means have the possibility of magnifying the field of view, and changing such a magnification over time, thus also varying the “FOV Workspace”, without physical movement of the viewing means. Thereby, as shown for example in FIG. 13 (in which the indications FOV(t1) and FOV (t2) refer precisely to the FOV at different instants in time), it is possible to enlarge the field of view without having to retract the camera, when the surgical instrument approaches the edges of the field of view FOV and/or is controlled in a position which is outside the current field of view. For example, such viewing means comprise digital viewing means suitable for robotic surgery and/or microsurgery. In accordance with an embodiment of the method, the aforesaid step of determining a position of the surgical instrument 170 with respect to the allowed space correlated to the viewing space comprises determining a current position of the surgical instrument 170 and/or the presence of the surgical instrument 170 in the allowed space correlated to the viewing space, based on digital data deriving from the viewing means.

According to another embodiment of the method, the aforesaid step of determining a position of the surgical instrument 170 with respect to the allowed space correlated to the viewing space comprises:
mapping the aforesaid allowed space correlated to the viewing space in a corresponding slave field-of-view workspace, in a slave reference coordinate system associated with the slave device;
determining the position of the surgical instrument 170 by means of respective position coordinates in the aforesaid slave reference coordinate system;
determining the position of the surgical instrument 170 with respect to the allowed space correlated to the viewing space based on a comparison between the aforesaid position coordinates and the aforesaid slave field-of-view workspace, in the slave reference coordinate system.

According to an implementation option of the aforesaid embodiment, the method further comprises the following steps:
defining, in the slave reference coordinate system SFO, a slave kinematic workspace 175, based on physical movement limits of the slave device and/or operating constraints not correlated to the viewing means;
defining, in the slave reference coordinate system, an effective slave workspace 200, corresponding to the intersection of the aforesaid slave field-of-view workspace and slave kinematic workspace 175.

In such a case, the step of controlling the slave device movement comprises controlling the slave device movement so that the movement of the surgical instrument 170 is allowed only if the surgical instrument 170 is located inside the aforesaid effective slave workspace 200, and that the movement of the surgical instrument 170, if allowed, is still confined within the aforesaid effective slave workspace 200.

More specifically, for example, a field-of-view slave workspace (or “FOV Slave Workspace”) can be defined as a workspace geometrically equivalent to the field-of-view slave workspace but transposed by means of a mapping function (e.g., rototranslation) in the reference system of the slave device.

Such an “FOV Slave Workspace” is intersected with the slave kinematic workspace 175 so as to ensure that it is therein, and therefore results in the aforesaid effective slave workspace 200, which can then be used by the various movement limitation algorithms.

According to an implementation option, the FOV slave workspace is contained within the slave kinematic workspace 175.

According to another implementation option, the field-of-view slave workspace is only partially contained within the slave kinematic workspace 175.

According to an implementation option, the effective slave workspace 200 is the intersection of the slave kinematic workspace 175 and the field-of-view slave workspace.

According to an implementation option, the effective slave workspace 200 is reduced and limited by the field-of-view slave workspace.

According to several possible implementations of the method, such a limitation can be done by means of a pure geometric intersection between the two convex geometries, or simplified in a parallelepiped or in a pyramid trunk inside such an intersection (thus calculated by the software, for example, to have an effective workspace 200 with a desired shape/usability).

As already noted, according to an embodiment of the method, the aforesaid step of determining whether the surgical instrument 170 is located inside or outside the viewing space is carried out minus a spatial tolerance e near the boundaries of the viewing space.

According to an implementation option, such a spatial tolerance e can depend on one or more factors such as speed, scale factor, magnification of the viewing medium, or others.

As already illustrated above, according to a preferred implementation option, such a spatial tolerance e defines an area larger than the viewing space area, and in another option an area smaller than the viewing space area.

According to an embodiment of the method, the aforesaid step of determining a position of the surgical instrument 170 with respect to the allowed space correlated to the viewing space is carried out cyclically and/or continuously in real time, to verify the position or presence of the surgical instrument 170 in the viewing space or in the effective slave workspace 200 in real time. According to an embodiment of the method, the aforesaid step of determining a position of the surgical instrument 170 with respect to the allowed space correlated to the viewing space is carried out cyclically and/or continuously in real time using and estimating the future pose of the surgical instrument with respect to said FOV Workspace starting from said master inputs, and from further context information such as the proximity to a target region, the movement history of the instrument in the last seconds.

According to an implementation example, the method is applied to a situation in which the field of view FOV is dynamic, and depends for example on the position of the viewing means (e.g., microscope), zoom, scale factor, and so on, while the kinematic workspace tends to be static and predefined.

According to an embodiment of the method, the aforesaid step of determining a position of the surgical instrument 170 with respect to the allowed space correlated to the viewing space comprises calculating and/or determining the position of a real point belonging to the surgical instrument or the position of a virtual point integral with the surgical instrument 170, based on images provided by said viewing system.

According to an implementation option, the aforesaid step of determining a position of the surgical instrument 170 comprises determining the position of a virtual control point 600 of the slave device (for example placed between the tips 171, 172 or “jaws” 171, 172 of the surgical instrument 170).

According to another implementation option, the aforesaid step of determining a position of the surgical instrument 170 comprises determining the position of at least one of the tips 171, 172 of the surgical instrument 170.

According to an implementation option, the aforesaid step of determining a position of the surgical instrument 170 comprises determining the position of at least one of the links of a hinged wrist (or “end-effector”) 177 included in the surgical instrument 170.

According to another implementation option, the aforesaid step of determining a position of the surgical instrument 170 comprises determining the position of a distal portion of a positioning shaft 179 or shaft 179 near the hinged wrist 177 of the surgical instrument 170.

In accordance with an embodiment of the method, the aforesaid step of determining the position of the surgical instrument 170 comprises determining the position of the surgical instrument 170 based on a nominal position of the slave device, in a workspace of the slave device defined in the slave reference coordinate system, controlled by the master device, or based on a nominal target pose of the slave device, in a workspace of the slave device defined in the slave reference coordinate system, corresponding to a respective pose of the master device in a workspace of the master device.

In accordance with an embodiment, the method comprises the further step of stopping the teleoperation of the robotic system, or exiting a teleoperation condition of the robotic system, if and when the presence of the surgical instrument is not detected within the allowed space correlated to the viewing space or in the effective slave workspace 200, or if and when said nominal position of the slave device is determined to be outside the allowed space correlated to the viewing space or outside the effective slave workspace 200.

In accordance with another embodiment, the method comprises the further step of allowing and/or enabling the movement of the surgical instrument 170 only when the presence of the surgical instrument is detected within the allowed space correlated to the viewing space or in the effective slave workspace 200, or only when the aforesaid nominal position of the slave device is determined to be within the allowed space correlated to the viewing space or within the effective slave workspace 200.

According to another embodiment, the method comprises the further step of allowing and/or enabling alignment operations between master device and slave device only when the presence of the surgical instrument is detected within the allowed space correlated to the viewing space or in the effective slave workspace 200, or only when the nominal position of the slave device is determined to be within the allowed space correlated to the viewing space or within the effective slave workspace 200. For example, the master device is a master device of the type not mechanically constrained to the operating console.

In accordance with another embodiment, in which the robotic system comprises a plurality of slave devices and respective surgical instruments, the method provides that movements or alignment operations between master device and slave device are allowed and/or enabled only for surgical instruments the presence of which is detected within the allowed space correlated to the viewing space or in the effective slave workspace 200, or only for surgical instruments for which the nominal position of the respective slave device is determined to be within the allowed space correlated to the viewing space or within the effective slave workspace 200.

According to an embodiment (shown for example in FIGS. 16 and 16bis), the method comprises the further step of modifying a slave nominal trajectory 606 of the slave device, corresponding to a respective master trajectory 601 of the master device 110, if and when the aforesaid slave nominal position is determined to be outside the viewing space (e.g., FOV) or outside the effective slave workspace 200, or if and when the aforesaid slave nominal trajectory 606 of the slave device exits the viewing space or the effective slave workspace 200.

In such a case, the aforesaid modifying step determines a modified slave target trajectory 607, such as to extend entirely within the viewing space (e.g., FOV) or the effective slave workspace 200, and/or in any case along the edges or limits imposed by the field-of-view workspace, as shown for example in FIG. 16.

In such a case, the method finally includes controlling the slave device so that it tracks and travel along said modified slave target trajectory.

According to an implementation option of the aforesaid embodiment, the step of modifying a nominal slave trajectory 606 of the slave device comprises stopping and/or freezing (“Freeze”) the surgical instrument 170 when the position thereof reaches the limits of the viewing space or the effective slave workspace 200). The action of stopping and/or freezing the surgical instrument 170 comprises locking all degrees of freedom of the surgical instrument 170, both translation and orientation degrees of freedom, and exiting the teleoperation.

According to another implementation option of the aforesaid embodiment, the aforesaid step of modifying a nominal slave trajectory 606 of the slave device comprises stopping and/or freezing the surgical instrument 170 when the position thereof reaches the limits of the allowed space correlated to the viewing space or the effective slave workspace 200. The action of stopping and/or freezing the surgical instrument 170 comprises, in this case, locking only a subset of the degrees of freedom of the surgical instrument 170, and allowing it to remain in teleoperation.

According to a particular implementation example, the aforesaid action of stopping and/or freezing the surgical instrument 170 comprises locking all the degrees of freedom of translation of the surgical instrument 170, leaving the degrees of freedom of distal orientation of the surgical instrument enabled, so that, close to the limits of the viewing space, the surgical instrument 170 or a control point 600 associated therewith does not follow the master device in translation and follows the master device in orientation.

According to an implementation example, the aforesaid action of stopping and/or freezing the surgical instrument 170 comprises locking the degree or degrees of freedom of translation associated with an exit direction from the boundary of the allowed space correlated to the viewing space, and keeping the other degrees of freedom of translation active.

In accordance with an implementation option of the method, when the movement imposed by the master device returns the slave device and the surgical instrument 170 in a direction or to a position within the allowed space correlated to the viewing space, the aforesaid stopping and/or freezing action is interrupted, so as to again allow the movement of the surgical instrument 170 according to all the degrees of freedom.

As noted above, some implementations provide that the aforesaid action of stopping and/or freezing (“Freeze”) the surgical instrument 170 comprises the action of locking all the actuated degrees of freedom or locking a part of the actuated degrees of freedom and/or in particular locking one or all of the degrees of freedom of related to position, leaving those of distal orientation enabled.

For example, close to the limits of the field of view FOV or the effective space 200, the surgical instrument 170 or a control point 600 associated with said surgical instrument does not follow the master device in translation and follows the master device in orientation. In such a situation, the slave device can be configured not to follow the master device in translation only in the direction out of the field of view FOV or the effective space 200, and to follow the master device 110 in translation when in the inwards-facing direction of the field of view FOV or the effective space 200.

In accordance with another implementation option of the aforesaid embodiment, the step of modifying a nominal slave trajectory of the slave device comprises reducing the movements of the slave device, with respect to the movements of the master device, according to a dynamically variable scale factor Fs, as the distance of the surgical instrument 170 decreases with respect to the limits of the viewing space or the effective slave workspace 200.

According to another implementation option of the aforesaid embodiment, the step of modifying a slave nominal trajectory of the slave device comprises decreasing the translational speed module of the slave device, in a direction orthogonal to the limits of the viewing space or the effective slave workspace 200, according to a transfer function dependent on the master device instantaneous speed and/or the master device instantaneous power or energy and/or the distance between a current position of the slave device and the limits of the viewing space or the effective slave workspace 200.

According to another implementation option of the aforesaid embodiment, the step of modifying a slave nominal slave of the slave device comprises decreasing the instantaneous power or energy imparted by the master device to the slave device according to a transfer function dependent on the master device instantaneous speed and/or the master device instantaneous power or energy and/or the distance between a current position of the slave device and the limits of said viewing space or said effective slave workspace 200.

In accordance with an embodiment of the method, the viewing space comprises the aforesaid field of view (FOV) of the viewing means, or a predefined subset of the field of view (FOV).

According to another embodiment, the method comprises the further step of defining limits or edges of the viewing space which in turn define upper and lower thresholds for the movements allowed to the slave device.

According to an implementation, the aforesaid limits or edges comprise a threshold perimeter on a plane XY which is orthogonal to a depth direction Z of the field of view FOV.

Such a threshold perimeter defines upper/lower thresholds for movements in the aforesaid plane XY and/or along orthogonal axes X, Y belonging to the plane XY. The threshold perimeter is calculated depending on the distance of the plane XY with respect to the viewing means.

According to another implementation option, the aforesaid limits or edges comprise, in addition to the threshold perimeter on a plane XY, also lower/upper thresholds along the axis of the depth direction Z of the field of view FOV.

In such a case, the aforesaid lower/upper thresholds along the axis of the depth direction Z are determined based on a good focusing of the viewing means, evaluated and calculated in real time using the data provided by the viewing means, or based on the field depth of the viewing means in a given configuration within a predefined focus acceptability range provided by the viewing means.

In an embodiment the field-of-view workspace limits are defined as a pyramid trunk defined by a Z as a combined function of X, Y and Z are calculated taking into account the intersecting workspace of a stereoscopic viewing system.

According to an implementation option, upper/lower thresholds are defined to avoid entering areas with excess disparity between the two points of view which lead to a blurred view for the operator.

According to an embodiment, the method further includes providing the operator with visual and auditory warnings when the device is close to the limits or edges of the viewing space or the effective slave workspace 200.

In accordance with an embodiment, the method comprises the further step of dynamically adjusting/varying the viewing space, by controlling the viewing means, e.g., by changing the zoom or adjusting the point of view, so as to improve or restore the view of the surgical instrument through the viewing means.

In particular, in an embodiment the robotic system coupled to the viewing system is capable of acting autonomously on the zoom by widening the viewing space (e.g., FOV) when an instrument reaches the limits of the field of view, thereby preventing a maneuver of the surgeon, which can be involuntary, from causing the exit of the instrument from the field of view.

In an embodiment, a first zoom value related to a first viewing space (e.g., first FOV) and a second zoom value related to a second viewing space (e.g., second FOV) are stored in which:
the aforesaid first zoom value is greater than the second zoom value;
the aforesaid first viewing space (e.g., first FOV is less than the aforesaid second viewing space (e.g., second FOV).

The self-adjustment of the zoom upon reaching the limits imposed by the viewing space (e.g., FOV) can vary between the aforesaid first zoom value and second zoom value and the related first FOV and second FOV.

For example, said varying zoom self-adjustment is an intermediate variation between the two values calculated and evaluated based on the target position of the instrument or upon reaching the limits, generating intermediate viewing spaces contained between the first and second viewing spaces, or is one of the two zoom values, and passes from one to the other when the instrument is outside or inside said first viewing space (e.g., FOV).

In an embodiment, an associated pedal allows switching the pressure, or maintenance, from said first zoom to second zoom.

In an embodiment, the aforesaid magnification is a portion of a high-resolution digital image and at least one of said first and second zoom values and first and second viewing spaces is a portion of said acquired digital image. In such an embodiment, the passage between the first zoom value and the second zoom value or vice versa does not include any mechanical movement of joints, lenses or microscope but only digital processing.

In an embodiment, said first workspace (e.g., first FOV) is a sub-portion of the image acquired by the viewing system and can change or simply move within the perimeter of the acquired image following the pose of the instrument and always keeping it within the aforesaid first viewing space. In such an embodiment, tracking the instrument and keeping it inside the FOV does not include any mechanical movement of joints, lenses or microscope but only digital processing.

According to an implementation option, the digital viewing system has an associated screen on which it projects the images.

According to an embodiment of the method, the movements of the slave device are stopped or inhibited minus a time tolerance, during which a slowed movement of the slave device is allowed even if the surgical instrument is outside the viewing space, so as to maintain directional movement consistency between master device and slave device.

Still referring to FIGS. 1-16, a robotic system 100 for medical or surgical teleoperation, included in the present invention, is described below.

Such a robotic system comprises at least one master device 110, adapted to be moved by an operator 150; at least one slave device comprising a surgical instrument 170 adapted to be controlled by the master device; viewing means configured to display to the operator 150 images and/or videos of a viewing space associated with a teleoperation area in which the surgical instrument 170 operates; and finally a control unit configured to control the slave device, during a teleoperation, based on movements of the master device.

The control unit is further configured to carry out the following actions:
determining a position of the surgical instrument 170 with respect to the aforesaid viewing space, to determine whether the surgical instrument 170 is located inside or outside an allowed space correlated to the viewing space;
controlling the movement of the slave device in a manner dependent on the determined position of the surgical instrument 170 with respect to the allowed space correlated to the viewing space, and so that the movement of the surgical instrument 170 is allowed only if the surgical instrument 170 is located inside the allowed space correlated to the viewing space.

According to several possible implementation options of the robotic system, the control unit is configured to carry out a method for controlling a slave device of a robotic system for medical or surgical teleoperation according to any one of the embodiments of such a method previously shown in this description.

As can be seen, the objects of the present invention as previously indicated are fully achieved by the method and system disclosed above by virtue of the features described above in detail.

Those skilled in the art may make changes and adaptations to the embodiments of the method and system described above or can replace elements with others which are functionally equivalent in order to meet contingent needs without departing from the scope of the following claims. Each of the features described as belonging to a possible embodiment can be implemented irrespective of the other embodiments described.

### LIST OF REFERENCE SIGNS

100 Robotic system for teleoperation
110. Master device
120 Viewing means, e.g., camera
150. Operator
170 Surgical instrument of the slave device
171, 172. Tip link (or “jaws”) of the slave surgical instrument
175. Slave kinematic workspace, or workspace of the slave device joints
175L. Left slave kinematic workspace
175R. Right slave kinematic workspace
177 Hinged wrist
179. Distal portion of rod or positioning shaft
200. Effective workspace, or useful workspace
600. Slave device control point
607. Modified target trajectory
FOV. Field of view
E. Tolerance
SFO. Global slave reference system
SF. Local slave reference system
SFL. Left local slave reference system
SFR. Right local slave reference system
t1, t2. First and second time instants
S-S. Surgical instrument roll axis

1. A method for controlling a slave device of a robotic system for medical or surgical teleoperation, wherein said robotic system comprises at least one master device adapted to be moved by an operator, at least one slave device, comprising a surgical instrument adapted to be controlled by the master device, and viewing means configured to display to the operator images and/or videos of a viewing space associated with a teleoperation area in which the surgical instrument operates, wherein the method comprises:
determining a position of the surgical instrument with respect to said viewing space, to determine whether the surgical instrument is located inside or outside an allowed space correlated to the viewing space;
controlling movement of the slave device in a manner dependent on the determined position of the surgical instrument with respect to the allowed space correlated to the viewing space, and so that movement of the surgical instrument is allowed only if the surgical instrument is located inside the allowed space correlated to the viewing space.

2. A method according to claim 1, wherein said allowed space correlated to the viewing space corresponds to the viewing space.

3. A method according to claim 1, wherein said allowed space correlated to the viewing space comprises the viewing space and further comprises an external surrounding extending with a spatial tolerance beyond boundaries of the viewing space.

4. A method according to claim 1, wherein said allowed space correlated to the viewing space comprises a subset of the viewing space, corresponding to the viewing space from which an internal surrounding extending with a spatial tolerance inside the boundaries of the viewing space is removed.

5. A method according to claim 1, wherein said viewing space is defined by:
a field of view of the viewing means, and/or
a predefined subset of the field of view of the viewing means, and/or
a field-of-view workspace, comprising a geometric volume, in a reference coordinate system of the robotic system, associated with said field of view, and/or
geometric limits of the field of view, comprising a boundary surface of said field-of-view workspace, in the reference coordinate system of the robotic system.

6. A method according to claim 1, wherein said determining a position of the surgical instrument with respect to the allowed space correlated to the viewing space comprises determining a current position of the surgical instrument and/or presence of the surgical instrument in the allowed space correlated to the viewing space based on digital data derived from the viewing means.

7. A method according to claim 1, wherein said determining a position of the surgical instrument with respect to the allowed space correlated to the viewing space comprises:
mapping said allowed space correlated to the viewing space in a corresponding slave field-of-view workspace, in a slave reference coordinate system associated with the slave device;
determining the position of the surgical instrument by respective position coordinates in said slave reference coordinate system;
determining the position of the surgical instrument with respect to the allowed space correlated to the viewing space based on a comparison between said position coordinates and said slave field-of-view workspace, in the slave reference coordinate system.

8. A method according to claim 7, further comprising:
defining, in the slave reference coordinate system a slave kinematic workspace based on physical movement limits of the slave device and/or operating constraints not correlated to the viewing means;
defining, in the slave reference coordinate system, an effective slave workspace, corresponding to an intersection of said slave field-of-view workspace and slave kinematic workspace; and
wherein the controlling the slave device movement comprises controlling the slave device movement so that the movement of the surgical instrument is allowed only if the surgical instrument is located inside said effective slave workspace, and so that the movement of the surgical instrument, if allowed, is still confined within said effective slave workspace.

9. A method according to claim 1, wherein said determining a position of the surgical instrument with respect to the allowed space correlated to the viewing space is performed cyclically and/or continuously in real time, to verify the position or presence of the surgical instrument in the viewing space or in the effective slave workspace in real time, and/or
wherein said determining a position of the surgical instrument with respect to the allowed space correlated to the viewing space comprises:
calculating and/or determining a position of a real point belonging to the surgical instrument or a position of a virtual point integral with the surgical instrument, and/or
calculating and/or determining a position of a virtual control point of the slave device, and/or
determining a position of at least one of the tips of the surgical instrument, and/or
determining a position of at least one of the links of a hinged wrist-included in the surgical instrument, and/or
determining a position of a distal portion of a positioning shaft proximate to the hinged wrist of the surgical instrument.

10. (canceled)

11. A method according to claim 7, wherein determining the position of the surgical instrument comprises determining the position of the surgical instrument based on a nominal position of the slave device, in a workspace of the slave device defined in the slave reference coordinate system, controlled by the master device, or based on a nominal target pose of the slave device, in a workspace of the slave device defined in the slave reference coordinate system, corresponding to a respective pose of the master device in a workspace of the master device.

12. A method according to claim 1, further comprising stopping the teleoperation of the robotic system, or exiting a teleoperation condition of the robotic system, if and when presence of the surgical instrument is not detected within the allowed space correlated to the viewing space or in the effective slave workspace, or if and when said nominal position of the slave device is determined to be outside the allowed space correlated to the viewing space or outside the effective slave workspace, or
further comprising allowing and/or enabling the movement of the surgical instrument only when the presence of the surgical instrument is detected within the allowed space correlated to the viewing space or in the effective slave workspace, or only when said nominal position of the slave device is determined to be within the allowed space correlated to the viewing space or within the effective slave workspace, or
further comprising allowing and/or enabling alignment operations between master device and slave device only when the presence of the surgical instrument is detected within the allowed space correlated to the viewing space or in the effective slave workspace, or only when said nominal position of the slave device is determined to be within the allowed space correlated to the viewing space or within the effective slave workspace, or
wherein the robotic system comprises a plurality of slave devices and respective surgical instruments, and wherein the movement or alignment operations between the master device and the slave device are allowed and/or enabled only for surgical instruments the presence of which is detected within the allowed space correlated to the viewing space or in the effective slave workspace, or only for surgical instruments for which the nominal position of the respective slave device is determined to be within the allowed space correlated to the viewing space or within the effective slave workspace.

13-15. (canceled)

16. A method according to claim 1, further comprising:
modifying a slave nominal trajectory of the slave device, corresponding to a respective master trajectory of the master device, if and when said slave nominal position is determined to be outside the allowed space correlated to the viewing space or outside the effective slave workspace, or if and when said slave nominal trajectory of the slave device exits the allowed space correlated to the viewing space or the effective slave workspace, wherein said modifying step determines a modified slave target trajectory, to extend entirely within the allowed space correlated to the viewing space or the effective slave workspace and/or along edges or limits imposed by the field-of-view workspace;
controlling the slave device so that the slave device tracks and travels along said modified slave target trajectory.

17. A method according to claim 12, wherein said modifying a slave nominal trajectory of the slave device comprises:
stopping and/or freezing the surgical instrument when the position of the surgical instrument reaches limits of said allowed space correlated to the viewing space or said effective slave workspace, wherein said action of stopping and/or freezing the surgical instrument comprises locking all the degrees of freedom of the surgical instrument, including both translation and orientation degrees of freedom, and exiting the teleoperation, or
wherein said modifying a slave nominal trajectory of the slave device comprises:
stopping and/or freezing the surgical instrument when the position of the surgical instrument reaches the limits of said allowed space correlated to the viewing space or said effective slave workspace, wherein said action of stopping and/or freezing the surgical instrument comprises locking only a subset of the degrees of freedom of the surgical instrument, and allowing to remain in teleoperation.

18. (canceled)

19. A method according to claim 17, wherein said action of stopping and/or freezing the surgical instrument comprises locking all the degrees of freedom of translation of the surgical instrument leaving the degrees of freedom of distal orientation of the surgical instrument enabled, so that, proximate the limits of the viewing space, the surgical instrument or a control point associated with said surgical instrument does not follow the master device in translation and follows the master device in orientation, and/or
wherein said action of stopping and/or freezing the surgical instrument comprises locking the degree or degrees of freedom of translation associated with an exit direction from the boundary of the allowed space correlated to the viewing space, and keeping the other degrees of freedom of translation active, and/or
wherein, when the movement imposed by the master device returns the slave device and the surgical instrument in a direction or to a position within the allowed space correlated to the viewing space, said stopping and/or freezing action is interrupted, so as to again allow the movement of the surgical instrument according to all the degrees of freedom.

20-21. (canceled)

22. A method according to claim 12, wherein said modifying a slave nominal trajectory of the slave device comprises:
decreasing a translational speed module of the slave device, in a direction orthogonal to the limits of said viewing space or said effective slave workspace, according to a transfer function dependent on a master device instantaneous speed and/or a master device instantaneous power or energy and/or a distance between a current position of the slave device and the limits of said viewing space or said effective slave workspace; and/or
decreasing the instantaneous power or energy imparted by the master device to the slave device according to a transfer function dependent on the master device instantaneous speed and/or the master device instantaneous power or energy and/or the distance between a current position of the slave device and the limits of said viewing space or said effective slave workspace; and/or
reducing the slave device movements, with respect to the master device movements, according to a dynamically varying scale factor, as a distance of the surgical instrument decreases with respect to the limits of said viewing space or said effective slave workspace.

23. A method according to or claim 7, wherein the viewing space comprises said field of view of the viewing means, or a predefined subset of the field of view, or said field of view workspace, or said effective slave workspace, wherein the method comprises further defining the limits or edges of the viewing space which in turn define upper and lower thresholds for the allowed movements of the slave device.

24. A method according to claim 23, wherein said limits or edges comprise a threshold perimeter on a plane orthogonal to a depth direction of the field of view, wherein said threshold perimeter defines upper/lower thresholds for movements in said plane and/or along orthogonal axes belonging to the plane, wherein said threshold perimeter is calculated depending on a distance of the plane with respect to the viewing means;
wherein said limits or edges comprise, in addition to the threshold perimeter on a plane, lower/upper thresholds along an axis of the depth direction of the field of view, wherein said lower/upper thresholds along the axis of the depth direction are determined based on a focusing of the viewing means, evaluated and calculated in real time using data provided by the viewing means, or based on the field depth of the viewing means in a given configuration.

25. (canceled)

26. A method according to claim 1, further comprising providing the operator with visual and auditory warnings when the device is close to the limits or edges of the viewing space or effective slave workspace; and/or
further comprising dynamically adjusting/varying the viewing space, by controlling the viewing means by changing zoom or adjusting a point of view, in order to improve or restore or not lose the view of the surgical instrument through the viewing means.

27. (canceled)

28. A method according to claim 27, further comprising storing a first zoom value related to a first viewing space and a second zoom value related to a second viewing space, wherein the first zoom value is greater than the second zoom value, and the first viewing space is smaller than the second viewing space,
wherein the self-adjustment of the zoom upon reaching the limits imposed by the viewing space is variable between said first zoom value and said second zoom value and related first viewing space and second viewing space, and/or
wherein said varying zoom self-adjustment is an intermediate variation between the two values calculated and evaluated based on a target position of the instrument or upon reaching the limits, generating intermediate viewing spaces contained between the first and second viewing spaces, or is one of the two zoom values, and/or
wherein said self-adjustment comprises switching from one zoom value to another zoom value when the instrument is outside or inside said first viewing space.

29. A robotic system for medical or surgical teleoperation, comprising:
at least one master device adapted to be moved by an operator;
at least one slave device comprising a surgical instrument adapted to be controlled by the master device;
viewing means configured to display to the operator images and/or videos of a viewing space associated with a teleoperation area in which the surgical instrument operates,
a control unit configured to control the slave device, during a teleoperation, based on master device movements,
wherein the control unit is further configured to:
determine a position of the surgical instrument with respect to said viewing space, to determine whether the surgical instrument is located inside or outside an allowed space correlated to the viewing space;
controlling the movement of the slave device in a manner dependent on the determined position of the surgical instrument with respect to the allowed space correlated to the viewing space, and so that the movement of the surgical instrument is allowed only if the surgical instrument is located inside the allowed space correlated to the viewing space.

30-56. (canceled)
